(19) United States
(12) Patent Application Publication
(10) Pub. No.: US 2002/0169466 A1
(43) Pub. Date: Nov. 14, 2002

(54) Medical grafting methods and apparatus

(75) Inventors: Alex Peterson, Maple Grove, MN (US); John Logan, Plymouth, MN (US); Jyue Boon Lim, Minneapolis, MN (US); William Swanson, St. Paul, MN (US); Jerry Grudem, St. Louis Park, MN (US)

Correspondence Address: FISH & NEAVE, 1251 AVENUE OF THE AMERICAS, 50TH FLOOR, NEW YORK, NY, 10020-1105, US

(73) Assignee: ST. JUDE MEDICAL ATG, INC.

(21) Appl. No.: 10/147,208

(22) Filed: May 14, 2002

Related U.S. Application Data
(60) Provisional application No. 60/290,701, filed on May 14, 2001.

(51) Int. Cl. A61B 17/08

(52) U.S. Cl. 606/153

(57) ABSTRACT

A connector for use in providing an anastomotic connection between two tubular body fluid conduits in a patient is provided. The connector is preferably a single, integral structure that can be cut from a tube. The connector has axially spaced portions that include “fingers” for engaging the two body fluid conduits. The connector also has members that have sharpened end portions that engage and penetrate the wall of one of the body fluid conduits. The fingers and sharpened members hold the two conduits together in a fluid-tight engagement. Apparatus for use in deploying a connector is also disclosed.

This application claims the benefit of U.S. provisional patent application No. 60/290,701, filed May 14, 2001, which is hereby incorporated by reference herein in its entirety.

### BACKGROUND OF THE INVENTION

This invention relates to medical grafting methods and apparatus and, more particularly, to methods and apparatus for use in making anastomotic connections between tubular body fluid conduits in a patient.

There are many medical procedures in which it is necessary to make an anastomotic connection between two tubular body fluid conduits in a patient. An anastomotic connection (or anastomosis) is a connection which allows body fluid flow between the lumens of the two conduits that are connected, preferably without allowing body fluid to leak out of the conduits at the location of the connection. As just one example of a procedure in which an anastomosis is needed, in order to bypass an obstruction in a patient's coronary artery, a tubular graft supplied with aortic blood may be connected via an anastomosis to the coronary artery downstream from the obstruction. The anastomosis may be between the end of the graft and an aperture in the side wall of the coronary artery (a so-called end-to-side anastomosis), or the anastomosis may be between an aperture in the side wall of the graft and an aperture in the side wall of the coronary artery (a so-called side-to-side anastomosis). The graft may be natural conduit, synthetic conduit, or a combination of natural and synthetic conduits. If natural conduit is used, it may be wholly or partly relocated from elsewhere in the patient (e.g., wholly relocated saphenous vein graft (“SVG”) or partly relocated internal mammary artery (“IMA”)). Alternatively, no relocation of the graft may be needed (e.g., a length of vein on the heart becomes a “graft” around an obstruction in an immediately adjacent coronary artery). More than one anastomosis may be needed. For example, a second anastomosis may be needed between an upstream portion of the graft conduit and the aorta or the coronary artery upstream from the obstruction in that artery. Again, this second anastomosis may be either an end-to-side anastomosis or a side-to-side anastomosis. Alternatively, no second, upstream anastomosis may be required at all (e.g., if the graft is an only-partly-relocated IMA).

The current most common technique for making an anastomosis is to manually suture the two tubular body fluid conduits together around an opening between them. Manual suturing is difficult and time-consuming, and the quality of the anastomosis that results is highly dependent on the skill of the person doing the suturing.

Various types of mechanical connectors have been developed to reduce or eliminate the need for suturing, but improvements are constantly sought for such mechanical connectors with respect to considerations such as ease and speed of use, ease of manufacture, strength and permanence of the resulting connection, etc.

Accordingly, it would be desirable to provide methods and apparatus for making anastomotic connections between tubular body fluid conduits in a patient.

### SUMMARY OF THE INVENTION

In accordance with the invention, a connector is provided for use in making an anastomotic connection between two tubular body fluid conduits in a patient, the connector being of substantially one-piece or unitary construction which extends annularly about a central longitudinal axis. The connector may include axially spaced first and second portions. The first and second portions may include “fingers” that expand radially out from the medial portion by, for example, unshielding the fingers during deployment of the connector in a patient's body. The first portion may include a plurality of members that have sharply pointed free end portions (e.g., for engaging and penetrating a graft conduit). In some embodiments, the connector may have a fixed diameter. For example, the connector may include a medial portion which is an annular structure having a fixed diameter. In other embodiments, the connector may be annularly enlargeable. For example, one or more of the first, second, and medial portions may be annularly enlargeable.

The connector is preferably constructed from nickel titanium alloy (“nitinol”) metal. The connector may be produced by removing selected material from a single, unitary metal tube. The machined tube may then be heat-shaped into approximately the geometry that the connector will assume after deployment.

The connector may typically be used to provide an anastomosis between an aperture in a side wall of a tubular graft conduit and an aperture in a side wall of the aorta in a coronary bypass procedure. An apparatus for forming an aperture in the side wall of the graft is disclosed. The apparatus may include a dilator for enlarging an incision made in the graft to the desired diameter.

An apparatus for deploying the connector so that it engages the graft and then engages the body tissue conduit is also disclosed. The apparatus may be used to load the graft onto the connector. In some embodiments, the distal end of the apparatus may be inserted into an open end of the graft to load the graft onto the connector (“front loading”). In other embodiments, the proximal end of the apparatus may be inserted into the aperture in the side wall of the graft to load the graft onto the connector (“back loading”).

In embodiments that use the back loading technique, the connector may include a plurality of members that extend radially out from the connector when the connector is constrained by the deployment apparatus. These members may assist in positioning the connector with respect to the aperture in the side wall of the graft.

After the apparatus for deployment has been inserted into the graft (e.g., using either the front loading or back loading techniques), the first portion fingers may be unshielded. The fingers may then radially expand such that the members having sharpened free end portions may engage the wall of the graft.

The assembly that includes the apparatus for deployment, the connector, and the graft may be inserted into an aperture in the side wall of the body tissue conduit. The assembly may be inserted such that the perimeter of the aperture in the side wall of the graft presses against the perimeter of the aperture in the side wall of the aorta. The second portion fingers may then be unshielded, such that the fingers radially expand and engage the inner surface of the body tissue conduit.

The apparatus for deployment may be removed from the graft through the open end of the graft, and the open end of the graft may be sealed off. This results in the formation of a side-to-side anastomosis between a graft and a body tissue conduit. In some embodiments, blood from the body tissue conduit may flow into the graft via the anastomotic connection. In other embodiments, blood may flow from the graft into the body tissue conduit via the anastomotic connection.

Further features of the invention, its nature, and various advantages will be more apparent from the accompanying drawings and the following detailed description of the preferred embodiments.

### BRIEF DESCRIPTION OF THE DRAWINGS

FIG. 1 is a simplified planar development of the structure of an illustrative embodiment of a connector constructed in accordance with the invention.

FIG. 2 is a simplified elevational view of the structure of the connector which is shown in planar development in FIG. 1.

FIG. 3 is another simplified elevational view of the structure of the connector which is shown in planar development in FIG. 1.

FIG. 4A is a simplified elevational view of the structure of FIGS. 1-3 with additional illustrative apparatus shown for use in delivering and deploying the structure of FIGS. 1-3 in a patient in accordance with the invention.

FIG. 4B is a simplified sectional view of the apparatus which is shown in a simplified elevational view in FIG. 4A.

FIG. 5 is a simplified elevational view of an illustrative graft conduit and illustrative apparatus for use in forming an aperture in the graft conduit showing an early stage in use of the apparatus in accordance with the invention.

FIG. 6 is a view similar to FIG. 5, but showing additional apparatus for use in creating an incision in the graft conduit, and showing a later stage in the use of the FIG. 5 apparatus in accordance with the invention.

FIG. 7 is a view similar to FIG. 6 showing the end result of using the FIG. 5 apparatus in accordance with the invention.

FIG. 8 is a simplified elevational view, partly in section, showing an early stage in the use of the FIG. 4A apparatus in accordance with the invention.

FIG. 9 is a view similar to FIG. 8 showing a later stage in use of the FIG. 4A apparatus in accordance with the invention.

FIG. 10 is a view similar to FIG. 9 showing a still later stage in use of the FIG. 4A apparatus in accordance with the invention.

FIG. 11 is a view similar to FIG. 10 showing a still later stage in use of the FIG. 4A apparatus in accordance with the invention.

FIG. 12 is a view similar to FIG. 11 showing the end result of using the FIG. 4A apparatus in accordance with the invention.

FIGS. 13A-13B are illustrative cross sectional views representing connectors constructed in accordance with the invention.

FIGS. 14-26 are views similar to FIG. 1 showing other illustrative embodiments of connectors constructed in accordance with the invention.

### DETAILED DESCRIPTION OF THE PREFERRED EMBODIMENTS

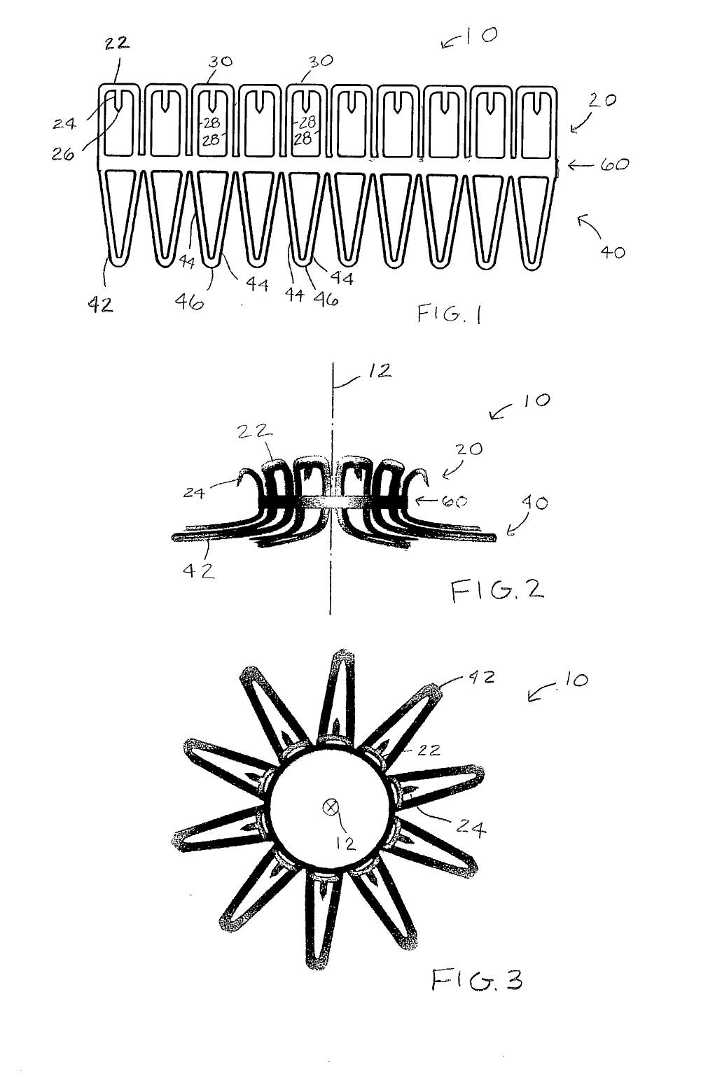

FIG. 1 shows a planar development of what is actually an integral, one-piece (unitary), annular connector 10. In particular, the left and right edges of connector 10 are actually joined to and integral with one another. Thus, the actual structure is as shown in FIGS. 2 and 3, although FIG. 1 is useful to more clearly reveal the details of various features of connector 10. A central longitudinal axis 12 about which connector 10 is annular is shown in FIGS. 2-3.

A particularly preferred material for connector 10 is nickel titanium alloy (“nitinol”) metal. Other examples of suitable materials include stainless steel, tantalum, tungsten, and platinum. Connector 10 may be advantageously produced by starting with a single, unitary-metal tube and removing selected material until only the structure shown in FIG. 1 remains. For example, laser cutting may be used to remove material from the starting tube in order to produce connector 10. After removing the material to form the structure shown in FIG. 1, the machined tube may be placed in a mold and heat-shaped into approximately the geometry that connector 10 will assume after deployment. For example, connector 10 may be heat-shaped into the geometry shown in FIGS. 2-3. The shape of connector 10 is retained after removing connector 10 from the mold due to the properties of nitinol.

Although connector 10 can be made in various sizes for various uses, a typical connector has a final inner diameter in the range from about 0.080 to about 0.180 inches to accommodate graft conduits having apertures in the range from about 2.5 to about 5.0 mm. For example, a connector having a final inner diameter of about 0.100 inches may accommodate a graft conduit having an aperture in its side wall in a range of about 3.0 to about 3.5 mm. A typical connector may be used in a patient's body tissue conduit (e.g., aorta) having a wall thickness in the range from about 1.5 to about 4.0 mm. A typical connector has a material thickness of about 0.006 inches. It will be understood, however, that these specific dimensions are only exemplary, and that any other dimensions can be used instead, if desired.

Connector 10 may be described as including axially spaced first and second portions 20 and 40, respectively. First portion 20 includes a plurality of annularly spaced cells 22. Cells 22 may also be referred to herein as “fingers.” A typical cell 22 includes a pair of annularly spaced longitudinal members 28. At the ends of members 28 that are farthest from second portion 40, the pair of members 28 are connected by member 30, which extends in the annular direction. At the end of members 28 that are closest to second portion 40, the pair of members 28 are connected to medial portion 60.

First portion 20 also includes a plurality of annularly spaced members 24 that are connected to cells 22 at annularly extending members 30. In this case, members 24 have free end portions 26 that are sharply pointed and that point toward second portion 40. Free end portions 26 may be sharpened, for example, by electropolishing each end portion until it attains the desired sharpness.

Second portion 40 includes a plurality of annularly spaced cells 42. Cells 42 may also be referred to herein as “fingers.” A typical cell 42 includes a pair of annularly spaced members 44. At the ends of members 44 that are farthest from first portion 20, the pair of members 44 are connected to one another at 46. At the end of members 44 that are closest to first portion 20, the pair of members 44 are connected to medial portion 60. In some embodiments, at medial portion 60, the ends of members 44 may be directly across from the ends of members 28. In other embodiments, at medial portion 60, the ends of members 44 may not be directly across from the ends of members 28, but rather cells 22 may be staggered in relation to cells 42. Also, connector 10 may include a different number of cells 22 than cells 42.

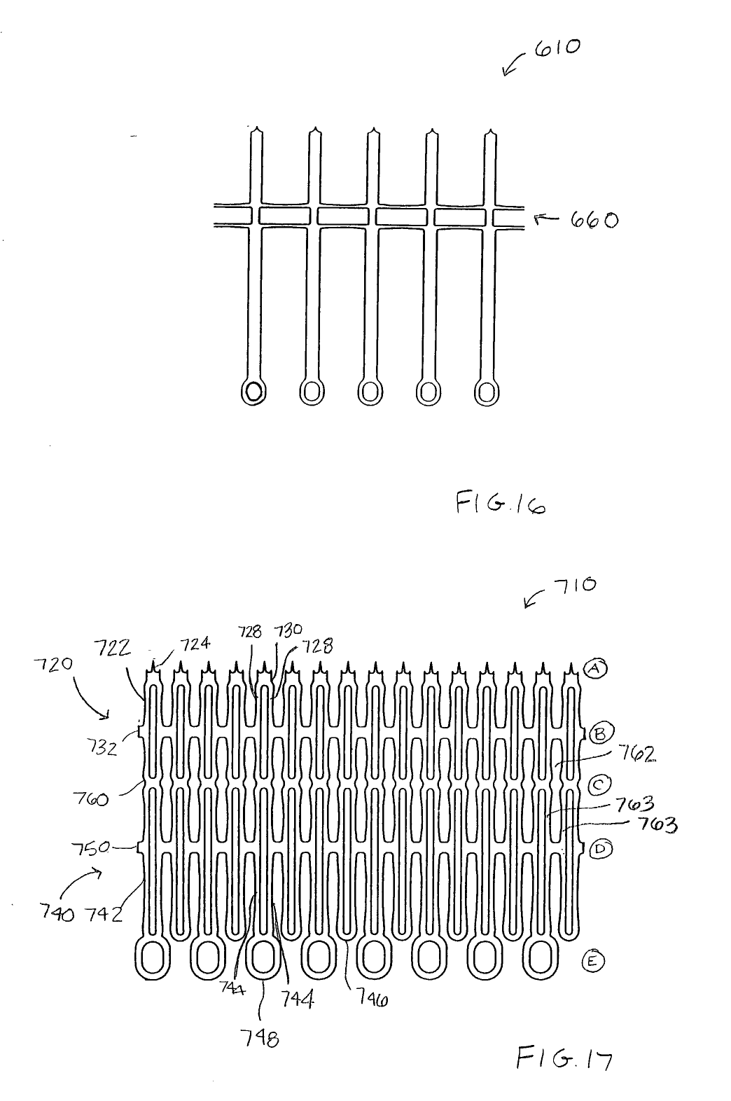

As shown in this example, connector 10 preferably has a fixed diameter. Specifically, medial portion 60 is an annular structure having a fixed diameter. In other examples, which are described below in reference to FIGS. 16-22 and 25-26, the connectors may be annularly enlargeable (e.g., one or more of the first, second, and medial portions of the connector may be annularly enlargeable).

As shown in FIGS. 2-3, fingers 22 and 42 may expand radially out from medial portion 60 (e.g., by unshielding the fingers during deployment of connector 10 in a patient's body). As described above, fingers 22 and 42 may expand to the configuration created by heat-shaping connector 10. The expansion of fingers 22 and 42 is preferably elastic.

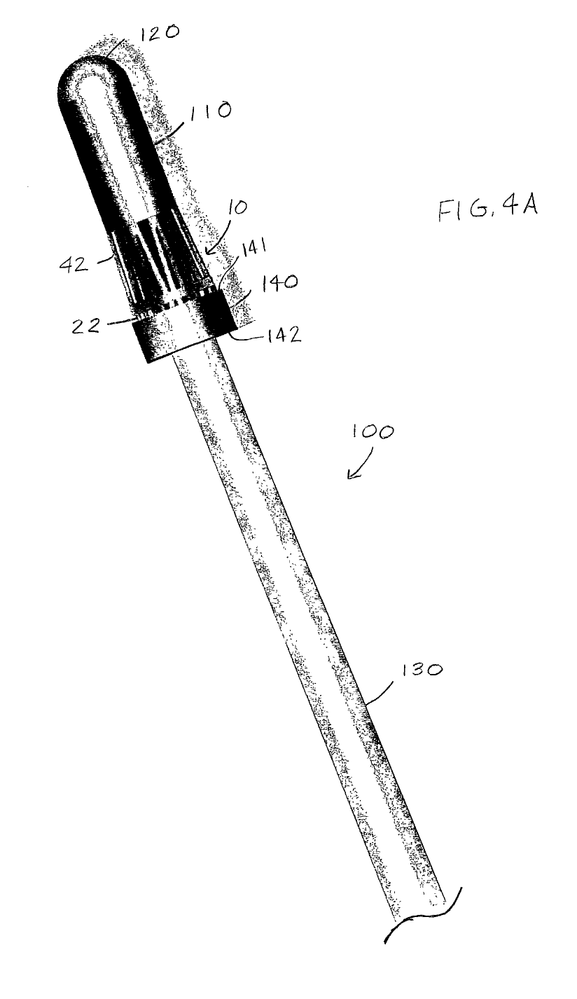

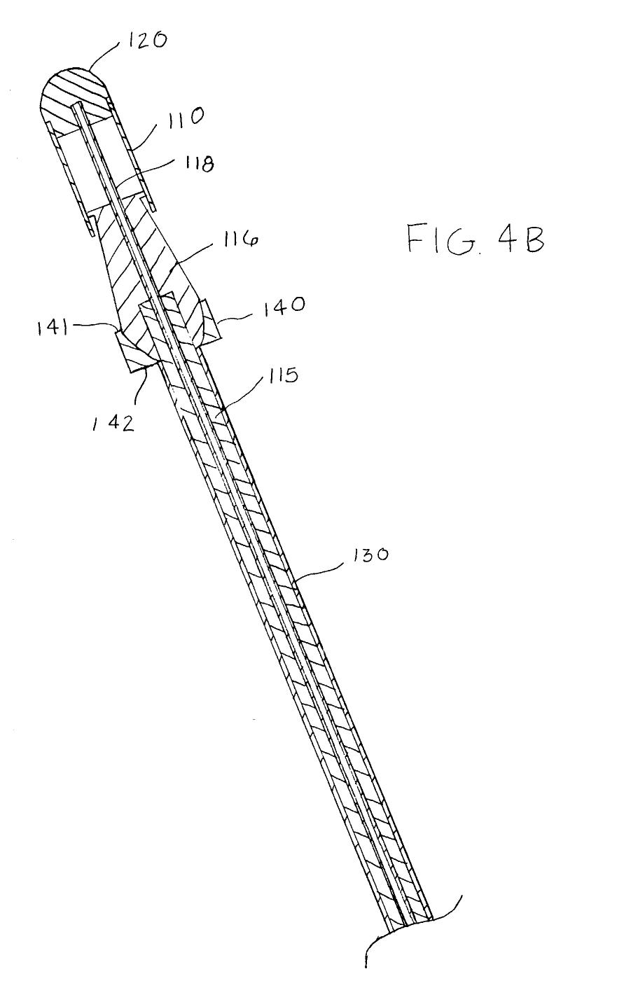

A typical use of connector 10 is to provide an anastomosis between an aperture in a side wall of a tubular graft conduit and an aperture in a side wall of the aorta in a coronary bypass procedure. An illustrative apparatus for deploying connector 10 so that it engages a tubular graft conduit and then engages a patient's body tissue conduit (e.g., aorta) is shown in FIGS. 4A and 4B. FIG. 4A is a simplified elevational view of apparatus 100 and connector 10 (FIGS. 1-3), and FIG. 4B is a simplified sectional view showing only apparatus 100 to further illustrate the relationships between the various elements of apparatus 100.

Apparatus 100 may include distal nose portion 110 having a hemispherical tip 120. The hemispherical shape of tip 120 is desirable to enable nose portion 110 to pass across a body conduit wall (e.g., an aortic wall) with minimal damage, with minimal force being required, and with no catching or snagging on the wall.

Nose portion 110 is connected to a tube 118 that extends proximally from the nose portion annularly within tube 115. Movement of tube 118 controls the position of nose portion 110 with respect to connector 10. In the position shown in this example, nose portion 110 is shielding at least a portion of fingers 42. Thus, fingers 42 may be described as being in a constrained configuration. In other words, fingers 42 are not in the radially expanded state shown in FIGS. 2-3.

Apparatus 100 includes a conical portion 116. Conical portion 116 is connected to tube 115. As described above, tube 115 is disposed annularly around tube 118. When nose portion 110 is in the position shown in FIG. 4A, fingers 42 may be “pinched” between conical portion 116 and the nose portion (i.e., in the constrained configuration). Movement of tube 118 relative to tube 115 in the distal direction unshields fingers 42 so that the fingers achieve the radially expanded state shown in FIGS. 2-3.

Apparatus 100 includes a tube 130 that extends proximally from tube 140. Tube 130 is disposed annularly around tube 115. Movement of tube 130 controls the position of tube 140 with respect to connector 10. In the position shown in this example, tube 140 is shielding at least a portion of fingers 22. Thus, fingers 22 may be described as being in a constrained configuration. In other words, fingers 22 are not in the radially expanded state shown in FIGS. 2-3. In the example shown in FIG. 4A, both sets of fingers 22 and 42 are constrained by apparatus 100 and, more particularly, by tube 140 and nose portion 110, respectively (e.g., for deployment of connector 10).

One or both of edges 141 and 142 of tube 140 may be rounded to facilitate passage of apparatus 100 within a graft conduit. Rounding one or both of edges 141 and 142 is desirable to avoid catching or snagging tube 140 on an opening or inner surface of the graft conduit.

The various elements of apparatus 100 may be constructed of a rigid material such as, for example, a metal.

Apparatus 100 may be used to load a tubular graft conduit onto connector 10. Prior to loading the graft onto connector 10, an aperture may be formed in the side wall of the graft. Preferably, such an aperture has a diameter that is about equal to the diameter of medial portion 60 of connector 10. In embodiments of connectors that are annularly enlargeable (e.g., such as the connectors shown in FIGS. 16-22 and 25-26), the diameter of the aperture in the side wall of the graft is preferably equal to the diameter of the connector in its expanded state.

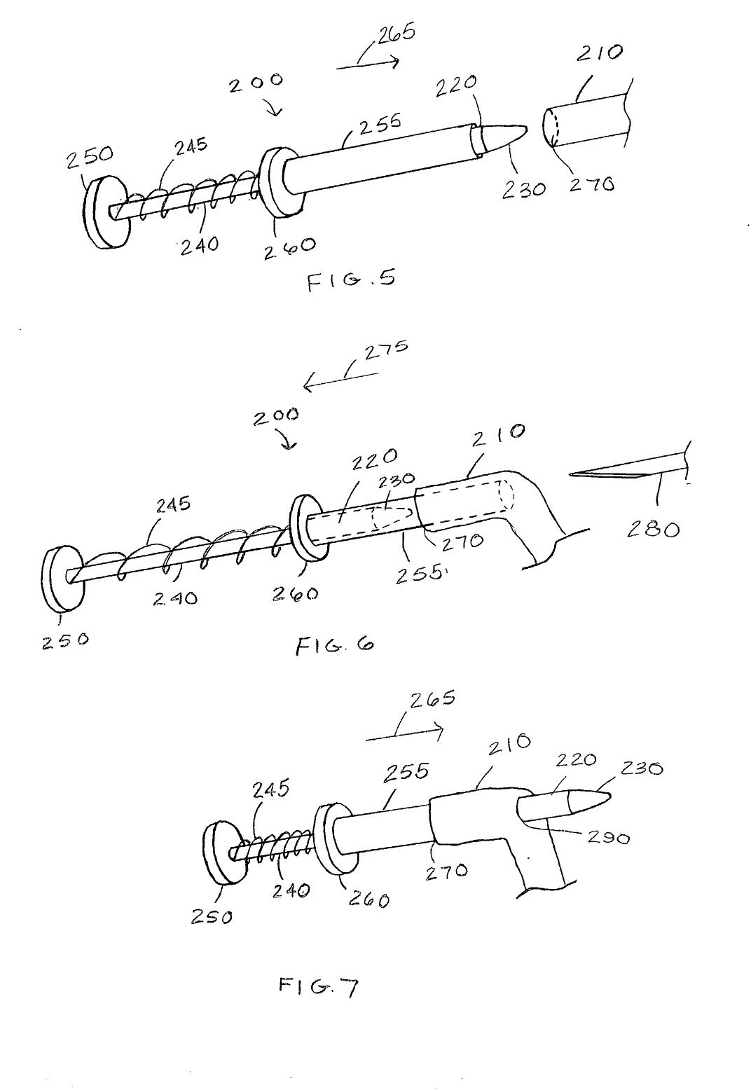

An illustrative apparatus 200 for forming an aperture in a side wall of a tubular graft conduit 210 is shown in FIG. 5. Apparatus 200 may be referred to as a “veinotomy tool” because of the veinotomy (i.e., aperture) it creates in graft 210. Graft 210 may be natural body tissue (e.g., a length of the patient's saphenous vein graft (“SVG”) harvested for use as a graft, a partly severed internal mammary artery (“IMA”), etc.), a synthetic graft (e.g., as shown in Goldsteen et al. U.S. Pat. No. 5,976,178 or published Patent Cooperation Treaty (“PCT”) patent publication No. WO 98/19632, published May 14, 1998, both of which are hereby incorporated by reference herein in their entireties), or a combination of natural and synthetic conduits (e.g., a length of natural conduit disposed substantially concentrically inside a length of synthetic conduit).

Apparatus 200 may include a dilator 220 having a nose portion 230. Dilator 220 may be attached to tube 240 so that the movement of tube 240 controls the movement of dilator 220. Disk 250 may be attached to tube 240 to facilitate the movement of tube 240. Apparatus 200 may also include tube 255 that is disposed annularly around dilator 220 and tube 240. Disk 260 may be attached to tube 255 to facilitate the movement of tube 255. A spring 245 may be disposed annularly around tube 240 between disks 250 and 260. Spring 245 may allow a physician to use apparatus 200 with one hand, so that the physician may hold other apparatus (e.g., a blade) with the other hand. In the configuration shown in FIG. 5, spring 245 may be slightly compressed.

Apparatus 200 may be advanced in the direction of arrow 265 such that nose portion 230 and at least a portion of tube 250 are received by open end 270 of graft 210. Nose portion 230 has a substantially conical outer surface. Nose portion 230 tapers such that its largest diameter (i.e., the diameter of the constant diameter portion of dilator 220) is about the same as the inner diameter of tube 255. Such tapering of nose portion 230 facilitates passage of tube 255 into open end 270 of graft 210, avoiding any catching or snagging on open end 270.

After nose portion 230 and at least a portion of tube 255 have been introduced into graft 210, dilator 220 may be retracted in the direction of arrow 275 as shown in FIG. 6. This leaves graft 210 disposed annularly around at least a portion of tube 255. When dilator 220 is retracted in the direction of arrow 275, spring 245 may return to a relaxed, or uncompressed, state.

Tube 255 may have an outer diameter that is equal to the outer diameter of tube 140 of deployment apparatus 100 (FIGS. 4A-4B). The matching outer diameters of tubes 255 and 140 may act as a gauge for sizing the aperture made in graft 210. For example, the outer diameter of tube 140 (FIG. 4), which is slightly larger than the outer diameter of connector 10 (FIGS. 1-3), may be indicative of the desired aperture size in graft 210. Thus, selecting an apparatus 200 having a tube 255 with an outer diameter equal to that of tube 140 (FIG. 4) results in formation of an aperture of the desired diameter.

Graft conduit 210 may be folded over the end of tube 255. Blade 280 may be advanced in the direction of arrow 275 to make an incision in graft 210.

Alternatively, the incision in graft 210 may be made from within the graft. In some embodiments, dilator 220 may be removed entirely from within tube 255 prior to making the incision. Other apparatus that includes a blade may be inserted into tube 255 and advanced in the direction of arrow 265 to make an incision in graft 210. In other embodiments, a blade may be “hidden” within dilator 220 (e.g., within nose portion 230). After dilator 220 has been retracted in the direction of arrow 275, the blade may be exposed. Dilator 220 and the exposed blade may then be advanced in the direction of arrow 265 to make an incision in graft 210.

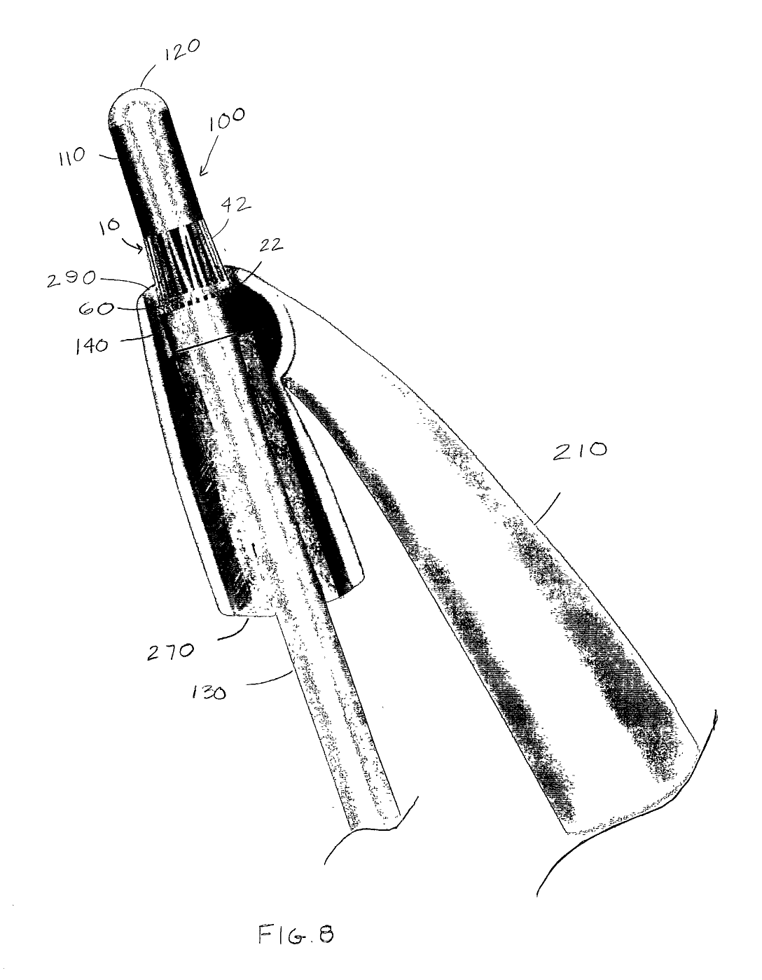

After making the incision in graft 210, dilator 220 may be advanced in the direction of arrow 265 as shown in FIG. 7. Dilator 220, and more particularly nose portion 230 gradually enlarges the incision made in the side wall of graft 210. The gradual taper of nose portion 230 enables the nose portion to pass across the side wall of graft 210 without snagging on the side wall. Dilator 220 is advanced until at least nose portion 230 has passed completely across the side wall of graft 210. As dilator 220 is advanced in the direction of arrow 265, spring 245 compresses. The passage of at least nose portion 230 across the side wall of graft 210 results in an aperture 290 of the desired size. For example, aperture 290 may be sized such that the diameter of the aperture at its “elastic limit” is equal to the diameter of medial portion 60 of connector 10 (FIGS. 1-3).

After forming aperture 290, apparatus 200 is removed from graft 210 by passing through open end 270.

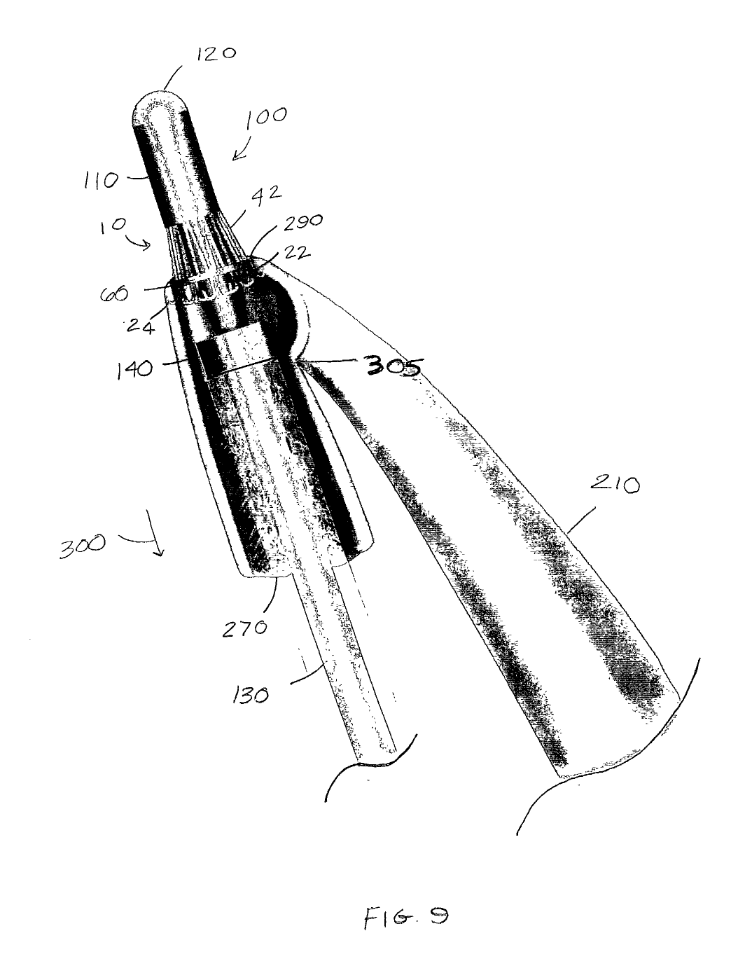

Graft 210 may then be placed annularly around tube 130 of apparatus 100, as shown in FIG. 8. For example, the assembly of apparatus 100 and connector 10 (FIG. 4A) may be inserted into open end 270 of graft 210. The assembly may then be advanced toward aperture 290 until at least a portion of medial portion 60 of connector 10 is disposed annularly within aperture 290. This technique of inserting the assembly of apparatus 100 and connector 10 into open end 270 of graft 210 may be referred to as “front loading,” since the distal end of apparatus 100 (i.e., nose portion 110) is the first portion of the apparatus to enter graft 210. In another example, the proximal end of apparatus 100 may be inserted into aperture 290 of graft 210. This technique may be referred to as “back loading,” since the proximal end of apparatus 100 is the first portion of apparatus 100 to enter graft 210. Such a technique may be used, for example, if the outer diameter of medial portion 60 is greater than the inner diameter of graft 210. In such a case, it is not possible for apparatus 100 to enter graft 210 through open end 270 because the outer diameter of medial portion 60 is greater than the inner diameter of the open end. Instead, the proximal end of apparatus 100 is inserted into aperture 290, and the assembly of apparatus 100 and connector 10 is advanced through graft 210 until at least a portion of medial portion 60 is disposed annularly within aperture 290. This “back loading” technique is described in further detail below in connection with FIGS. 23-26.

As shown in FIG. 9, tube 130 may be retracted in the direction of arrow 300 such that tube 140 no longer constrains fingers 22. Fingers 22 may then radially expand such that members 24 engage and penetrate the wall of graft 210 around the perimeter of aperture 290. The sharpened free end portions 26 (FIG. 1) facilitate penetration of graft 210 by members 24. Each member 24 may penetrate the wall of graft 210 until its movement is resisted by the connection between member 24 and horizontal member 30 (FIG. 1). The joint between member 24 and horizontal member 30 (FIG. 1) acts as a “stop,” restricting the movement of members 24.

During the deployment of members 24, fold 305 of graft conduit 210 may reside underneath of tube 140 (i.e., at the junction between tubes 140 and 130). This may prevent the tissue at fold 305 from interfering with the deployment of members 24. For example, in the absence of tube 140, the tissue at fold 305 may be engaged by one or more members 24, resulting in a partial or total occlusion of graft 210.

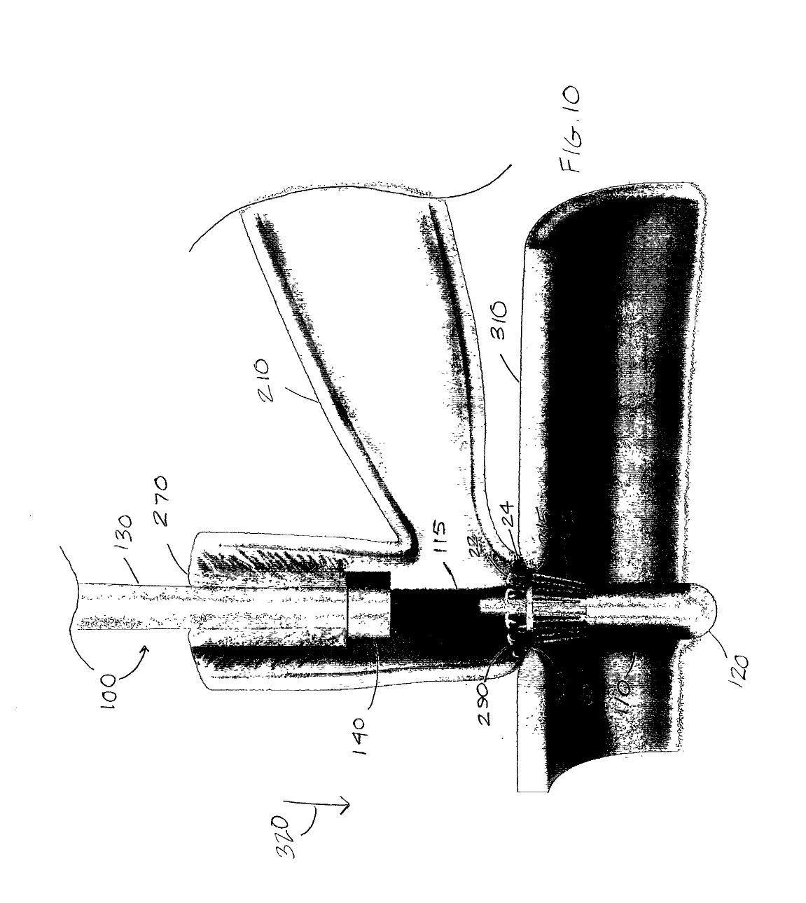

As shown in FIG. 10, nose portion 110 may be inserted into aperture 315 of a patient's tubular body tissue conduit 310 (e.g., a patient's aorta requiring a bypass graft) to connect graft 210 to the body tissue conduit. Aperture 315 may be formed, for example, by using a cutting catheter to cut through body tissue conduit 310 at the desired anastomosis site (e.g., as in published PCT patent publication No. WO 99/38441, published Aug. 5, 1999, which is hereby incorporated by reference herein in its entirety). The natural elastic recoil of the side wall of body tissue conduit 310 seals aperture 315 around nose portion 110 so that there is little or no body fluid (e.g., blood) leakage out of the body conduit via aperture 315. Nose portion 110 is gradually forced in the direction shown by arrow 320 until the perimeter of aperture 290 on the outer surface of the side wall of graft 210 presses against the perimeter of aperture 310 on the outer surface of the side wall of body tissue conduit 310, thereby forming a seal between the two apertures.

Tube 118 (FIG. 4A) may be moved in the direction of arrow 320 such that fingers 42 are released from the confines of nose portion 110, and are no longer “pinched” between the nose portion and conical portion 116 (FIG. 4A). Fingers 42 may then radially expand and achieve the configuration shown in FIG. 11.

After fingers 42 have radially expanded, apparatus 100 may be withdrawn from body tissue conduit 310 and graft 210. For example, apparatus 100 may be pulled in the direction of arrow 325 such that the apparatus exits the graft via open end 270. This leaves aperture 290 in the side wall of graft 210 connected to aperture 315 in the side wall of body tissue conduit 310 by connector 10.

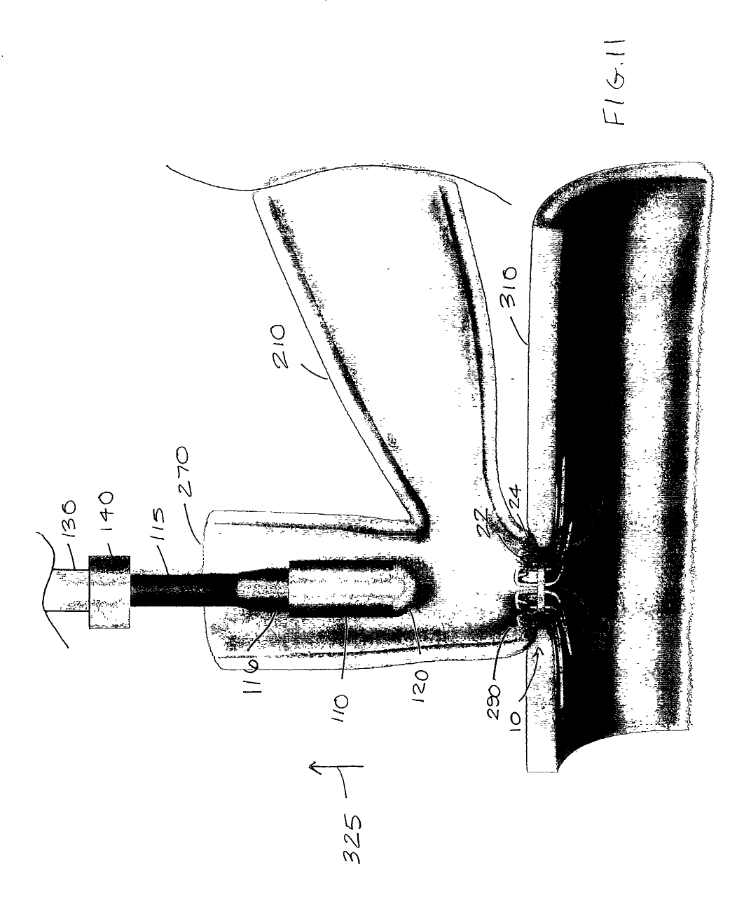

After apparatus 100 has been removed from graft 210, open end 270 of the graft may be sealed off using a closure 330 as shown in FIG. 12. In this example, connector 10 provides a side-to-side anastomosis between graft 210 and body tissue conduit 310. Body fluid from body tissue conduit 310 is able to flow into graft 210 via this connection (e.g., blood is able to flow from the aorta into the graft conduit). It will be appreciated that the direction of flow is entirely arbitrary, and that in another application of the invention the blood flow direction could be opposite to that just described.

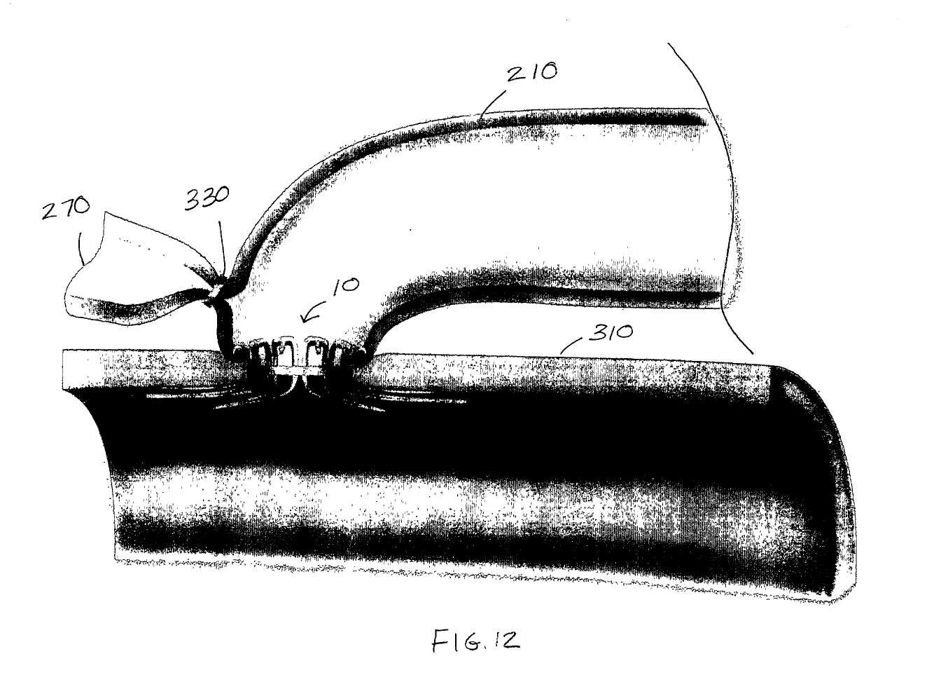

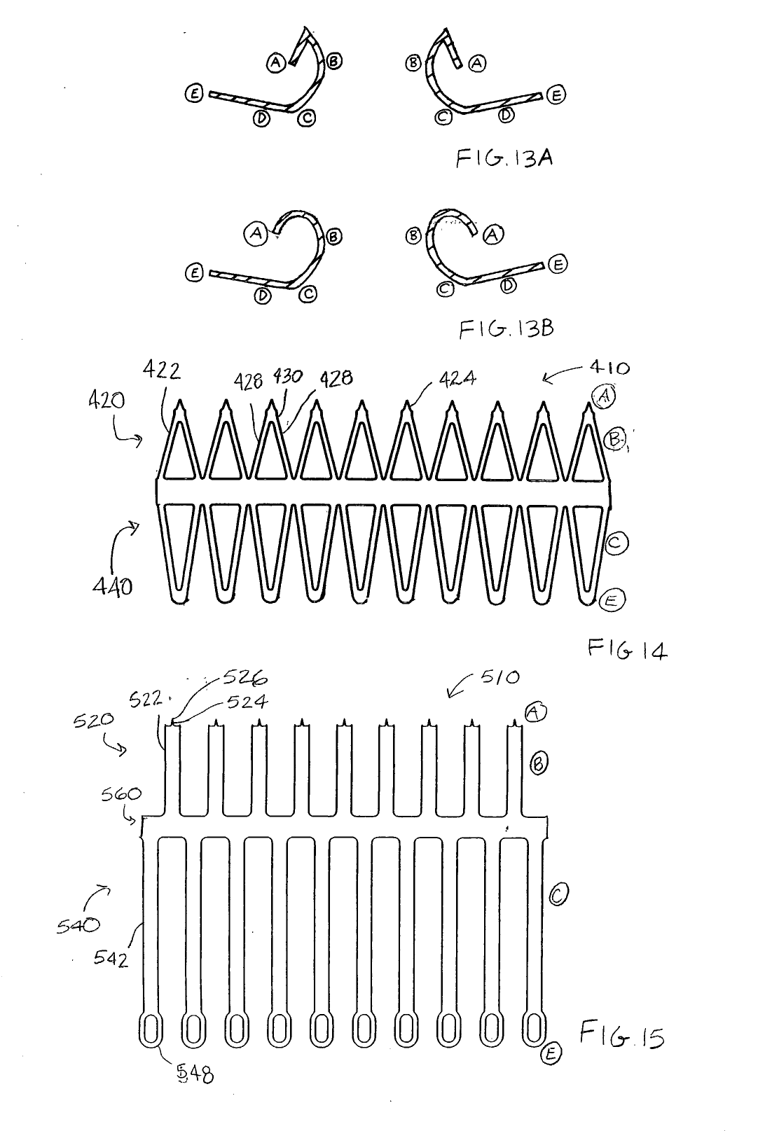

Illustrative cross-sectional representations of connectors constructed in accordance with the invention are shown in FIGS. 13A-13B. These cross-sectional representations are provided to simplify the description of the illustrative embodiments of connectors shown in the following FIGS. 14-26.

The cross-sectional representation shown in FIG. 13A is applicable to embodiments of connectors in which the members that engage the graft conduit (e.g., members 24 of connector 10, shown in FIG. 1) point toward the second portion of the connector (e.g., second portion 40 of connector 10, shown in FIG. 1). In such embodiments, the connector is formed such that the members that engage the graft conduit are bent outward from the connector structure, as shown in FIG. 13A.

The cross-sectional representation shown in FIG. 13B is applicable to embodiments of connectors in which the members that engage the graft conduit (e.g., members 424 of connector 410, shown in FIG. 14) point away from the second portion of the connector (e.g., second portion 440 of connector 410, shown in FIG. 14). In such embodiments, the connector is formed such that the members that engage the graft conduit are “curled” over, as shown in FIG. 13B. The result is a smooth curve between points A and B on the cross-sectional representation shown in FIG. 13B, in contrast to the “joint” between points A and B on the cross-sectional representation shown in FIG. 13A.

As specified in the description below, each of the connectors shown in FIGS. 14-26 may correspond to one or the other of the cross-sectional representations shown in FIGS. 13A and 13B.

As shown, points A, B, C, D, and E are represented along the cross-sectional representations shown in FIGS. 13A and 13B. Some or all of these points may be shown on the simplified planar developments of the connectors shown in FIGS. 14-26 to demonstrate the location of the points in relation to this illustrative cross-section.

The connectors in FIGS. 14-26 (shown in simplified planar development) are all suitable for use with the apparatus and methods shown in FIGS. 4A-12 to provide an anastomosis between an aperture in a side wall of a graft conduit and an aperture in a side wall of a patient's body tissue conduit. The connectors in FIGS. 14-26 are of a similar size as connector 10 (FIGS. 1-3), and the connectors are constructed of the same materials as connector 10. The differences between the embodiments of connectors shown in FIGS. 14-26 and connector 10 are made apparent in the description that follows, in conjunction with the cross-sectional representations shown in FIGS. 13A-13B.

An illustrative embodiment of a connector 410 in accordance with the invention is shown in FIG. 14. Connector 410 is substantially similar to connector 10 (FIGS. 1-3). However, fingers 422 of first portion 420 may have a triangular shape, instead of the rectangular shape of fingers 22 of first portion 20 (FIG. 1). For example, cells 422 may include annularly spaced members 428 that are connected at 430. In addition, members 424 that engage the graft conduit point away from second portion 440, resulting in a cross-section as shown in FIG. 13B. Points A, B, C, and E shown on connector 410, in conjunction with the cross-sectional representation of a connector in FIG. 13B, demonstrate the approximate geometry that connector 410 will assume after deployment.

An illustrative embodiment of a connector 510 in accordance with the invention is shown in FIG. 15. Connector 510 may be described as including axially spaced first and second portions 520 and 540, respectively. First portion 520 may include a plurality of annularly spaced members 522. First portion 520 may also include a plurality of annularly spaced members 524 that are connected to members 522 and point away from second portion 540. Members 524 may each have a free end portion 526 that is sharply pointed.

Second portion 540 may include a plurality of annularly spaced members 542. Each member 542 may include an attachment portion 548. Attachment portion 548 may facilitate attachment of connector 510 to a mold for heat-shaping. For example, the mold may include a pin around which attachment portion 548 may reside. (The connectors shown in the following FIGS. 16-22 and 25-26 may also include attachment portions which function in the same way as attachment portion 548.)

Members 522 and 542 may be connected to medial portion 560. In some embodiments, members 522 and 542 may be spaced annularly around medial portion 560 such that no member is directly across from another member. In other embodiments, members 522 and 542 may be spaced annularly around medial portion 560 such that the members are directly across from one another.

Connector 510 has a cross-section as shown in FIG. 13B because members 524 point away from second portion 540. Points A, B, C, and E shown on connector 510, in conjunction with the cross-sectional representation of a connector in FIG. 13B, demonstrate the approximate geometry that connector 510 will assume after deployment.

The connectors described above in connection with FIGS. 14-15 each have a fixed diameter, as does connector 10 (FIGS. 1-3). The connectors described in connection with the following FIGS. 16-21 are annularly expandable, thereby expanding from an initial diameter to a final, deployed diameter. One or more of the first, second, and medial portions of the following connectors may be annularly enlargeable.

An illustrative embodiment of a connector 610 in accordance with the invention is shown in FIG. 16. Connector 610 is substantially similar to connector 510 (FIG. 15). However, medial portion 660 differs from medial portion 560 of connector 510 (FIG. 15). Medial portion 660 of connector 610 may be referred to as having a “bow tie” design. For example, when connector 610 is constrained for deployment (e.g., by apparatus 100 of FIG. 4A), medial portion 660 resembles a chain of bow-tie-shaped cells.

An illustrative embodiment of a connector 710 in accordance with the invention is shown in FIG. 17. Connector 710 may be described as including axially spaced first and second portions 720 and 740, respectively.

Connector 710 is formed in such a way that it is annularly enlargeable (e.g., by heat-shaping the connector on a mold to a shape such as that shown in FIG. 13B). The annular expandability of connector 710 is provided by making the connector with a plurality of annularly adjacent, annularly enlargeable cells. For example, a typical cell 722 includes annularly spaced, but adjacent, members 728. The axially spaced ends of this pair of members are connected to one another at 730 and 760. Annularly adjacent cells 722 are connected to one another (e.g., as at 732) at locations which are axially medial to their axial end connections 730 and 760. In this way connector 710 is annularly enlargeable by annularly enlarging each of the above-mentioned cells 722.

The annular expandability of connector 710 is also provided by making the connector with a plurality of annularly adjacent, annularly enlargeable cells 742. For example, a typical cell 742 includes annularly spaced, but adjacent, members 744. The axially spaced ends of this pair of members are connected to one another at 746 and 760, or at 748 and 760. For example, attachment portion 748 may be at the connection between members 744 for every other cell 742. Annularly adjacent cells 742 are connected to one another (e.g., as at 750) at locations which are axially medial to their axial end connections 746 and 760, or 748 and 760. In this way connector 710 is also annularly enlargeable by annularly enlarging each of the above-mentioned cells 742.

In addition to cells 722 and 742 that are described above, connector 710 includes other, similarly annularly enlargeable cells 762 that are axially and annularly offset from the first-described cells. A representative one of these other cells 762 includes annularly spaced, but adjacent, longitudinal members 763, the axially spaced ends of which are connected at 732 and 750. (It should be noted that part of each member 763 is common with a part of each member 728 and 744.) Thus again the structure is annularly enlargeable by annularly enlarging cells 762.

Connector 710 has a cross-section as shown in FIG. 13B because members 724 point away from second portion 740. Points A, B, C, D, and E shown on connector 710, in conjunction with the cross-sectional representation of a connector in FIG. 13B, demonstrate the approximate geometry that connector 710 will assume after deployment.

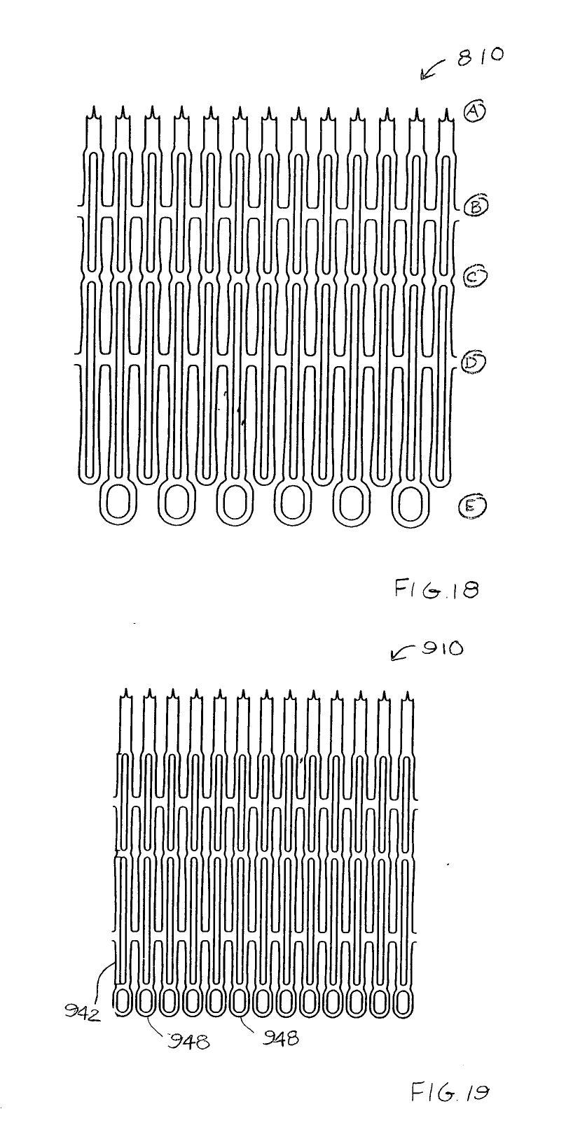

An illustrative embodiment of a connector 810 in accordance with the invention is shown in FIG. 18. Connector 810 is substantially similar to connector 710 (FIG. 17). However, the distance between points A and B on connector 810 may be greater than the distance between points A and B on connector 710 (FIG. 17). By increasing the distance between points A and B, points B, C, D, and E move further out along the body of the connector. Thus, in its expanded configuration (FIG. 13B), the diameter of points D of connector 810 is greater that the diameter of points D of connector 710 (FIG. 17).

An illustrative embodiment of a connector 910 in accordance with the invention is shown in FIG. 19. Connector 910 is substantially similar to connector 810 (FIG. 18). However, connector 910 may have an attachment portion 948 at the end of each cell 942.

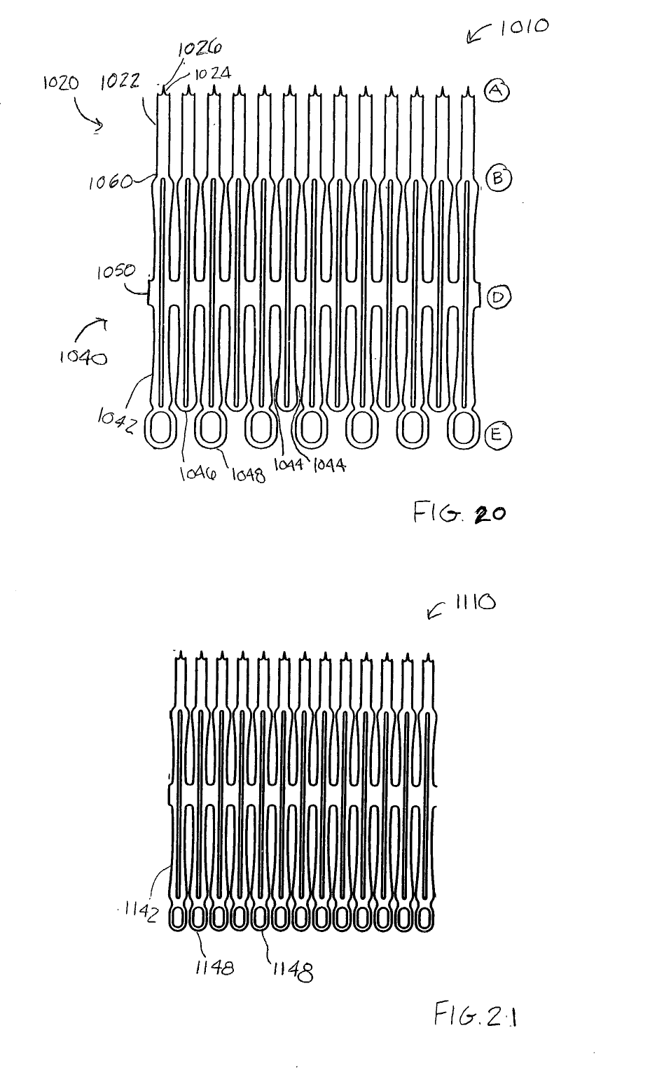

An illustrative embodiment of a connector 1010 in accordance with the invention is shown in FIG. 20. Connector 1010 may be described as including axially spaced first and second portions 1020 and 1040, respectively. First portion 1020 may include a plurality of annularly spaced members 1022. First portion 1022 may also include a plurality of members 1024 connected to members 1022 and pointing away from second portion 1040. Each member 1024 may have a free end portion 1026 that is sharply pointed.

Second portion 1040 may include a plurality of annularly spaced cells 1042. A typical cell 1042 includes annularly spaced, but adjacent, longitudinal members 1044. The axially spaced ends of this pair of members are connected to one another at 1046 and 1060, or at 1048 and 1060. For example, attachment portion 1048 may be at the connection between members 1044 for every other cell 1042. Cells 1042 may be connected to annularly adjacent cells 1042 by struts 1050.

As shown, connector 1010 may be described as being a hybrid between connectors such as connector 510 (FIG. 15) and connector 710 (FIG. 17). For example, connector 1010 has members 1022 that are similar to members 522 of connector 510, and connector 1010 has cells 1042 that are similar to cells 742 of connector 710 (FIG. 17).

Connector 1010 has a cross-section as shown in FIG. 13B because members 1024 point away from second portion 1040. Points A, B, D, and E shown on connector 1010, in conjunction with the cross-sectional representation of a connector in FIG. 13B, demonstrate the approximate geometry that connector 1010 will assume after deployment.

An illustrative embodiment of a connector 1110 in accordance with the invention is shown in FIG. 21. Connector 1110 is substantially similar to connector 1010 (FIG. 20). However, connector 1110 may have attachment portions 1148 at the end of each cell 1142.

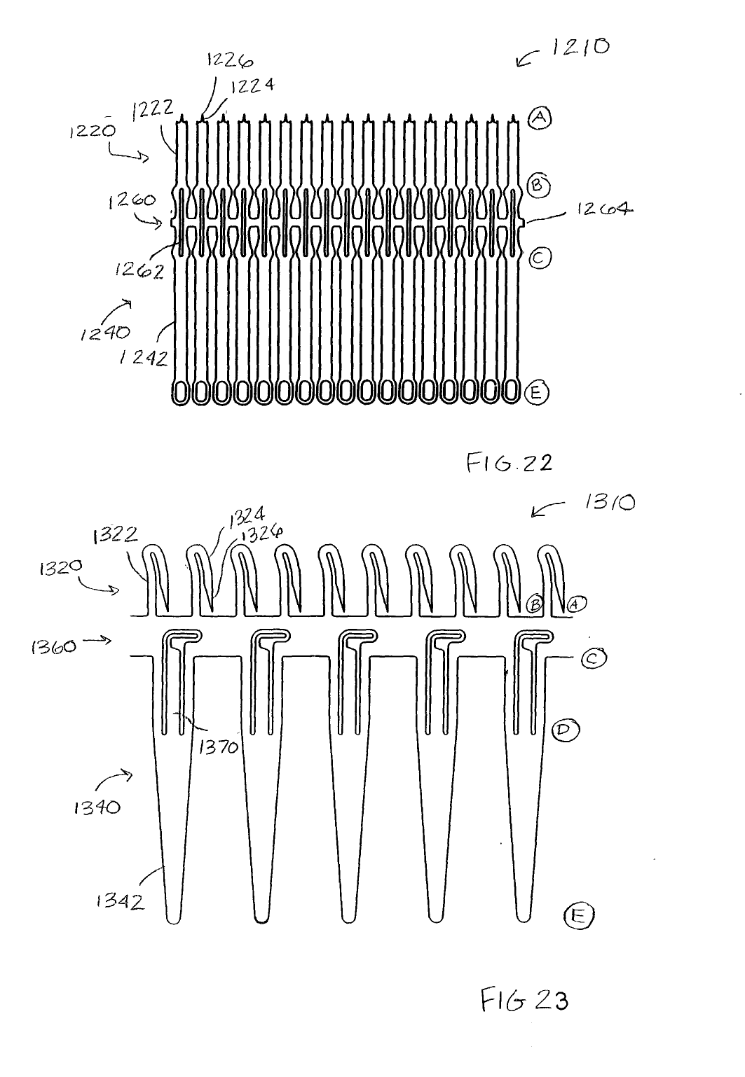

An illustrative embodiment of a connector 1210 in accordance with the invention is shown in FIG. 22. Connector 1210 may be described as including axially spaced first and second portions 1220 and 1240, respectively. First portion 1220 may include a plurality of annularly spaced members 1222. First portion 1220 may also include a plurality of members 1224 that are connected to members 1222 and that point away from second portion 1240. Members 1224 may each have a free end portion 1226 that is sharply pointed. Second portion 1240 may include a plurality of annularly spaced members 1242. Members 1242 may each include an attachment portion 1248.

Members 1222 and 1242 may be connected to medial portion 1260. In this example (in contrast to, for example, connector 510 of FIG. 15), medial portion 1260 may include expansion cells 1262. Each expansion cell 1262 is connected to annularly adjacent expansion cells 1262 by struts 1264.

Connector 1210 has a cross-section as shown in FIG. 13B because members 1224 point away from second portion 1240. Points A, B, C, and E shown on connector 1210, in conjunction with the cross-sectional representation of a connector in FIG. 13B, demonstrate the approximate geometry that connector 1210 will assume after deployment.

The connectors shown in the following FIGS. 23-26 are all connectors that, in conjunction with apparatus 100 (FIG. 4A), are back loaded into a graft conduit, as described above in reference to FIG. 8. Such a technique may be used, for example, if the outer diameter of the connector is greater than the inner diameter of the graft conduit. Each of the following embodiments of connectors have members that act as “stops” to assist in positioning the connector with respect to the aperture in the graft (see, for example, FIG. 8). These “stops” slightly change the approximate geometry that the connector will assume after deployment that is shown in FIGS. 13A and 13B. For example, from at or around the area between points C and D on the cross-sectional representations shown in FIGS. 13A and 13B, a “stop” would extend toward the inner diameter of the connector. However, the remainder of the cross-sectional representation is the same.

An illustrative embodiment of a connector 1310 in accordance with the invention is shown in FIG. 23. Connector 1310 may be described as including axially spaced first and second portions 1320 and 1340, respectively. First portion 1320 includes a plurality of annularly spaced members 1322. First portion 1320 also includes a plurality of spaced members 1324 that are connected to members 1322 and that point toward second portion 1340. Members 1324 may each have a sharply pointed free end portion 1326. At the ends of members 1322 that are closest to second portion 1340, members 1322 are connected to medial portion 1360.

Second portion 1340 includes a plurality of annularly spaced members 1342. At the ends of members 1342 that are closest to first portion 1320, members 1342 are connected to medial portion 1360.

Second portion 1340 includes a plurality of annularly spaced members 1370, described above as “stops.” Members 1370 may point toward first portion 1320. When connector 1310 is constrained by deployment apparatus such as apparatus 100 (FIG. 4A), members 1370 may extend radially out from the constrained connector. Thus, when connector 1310 is back loaded into a graft conduit, members 1370 may position the connector with respect to the aperture in the side wall of the graft conduit by coming into contact with the outer surface of the side wall (see, for example, FIG. 8 for the proper positioning of a connector in an aperture).

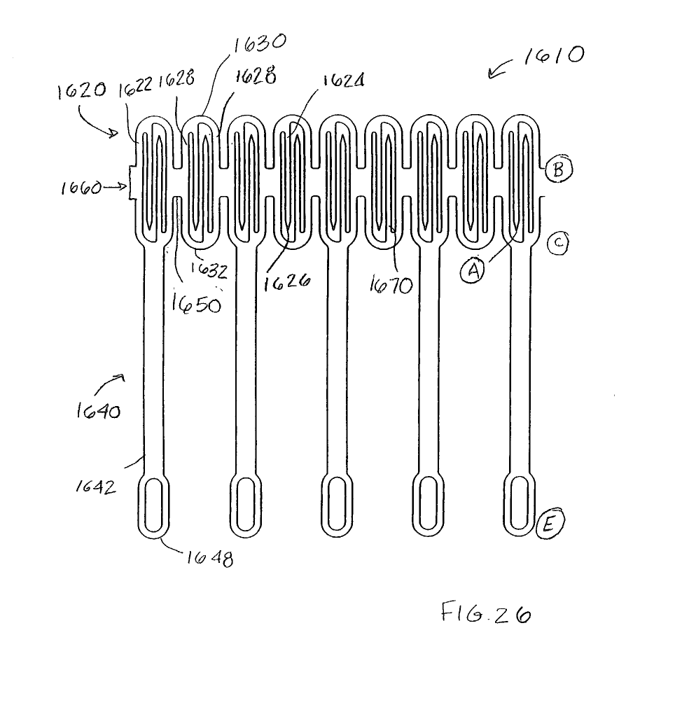

As shown in this example, connector 1310 is an annular structure having a fixed diameter (i.e., medial portion 1360 has a fixed diameter). Examples of connectors that may be back loaded into a graft conduit and that are annularly expandable are described below in connection with FIGS. 25 and 26.

Connector 1310 has a cross-section that is similar to that shown in FIG. 13A because members 1324 point toward second portion 1340. Points A, B, C, D, and E shown on connector 1310, in conjunction with the cross-sectional representation of a connector in FIG. 13A, demonstrate the approximate geometry that connector 1310 will assume after deployment. As shown, point A is located at free end portion 1326 of member 1324, while point B is located at the connection between member 1322 and medial portion 1360.

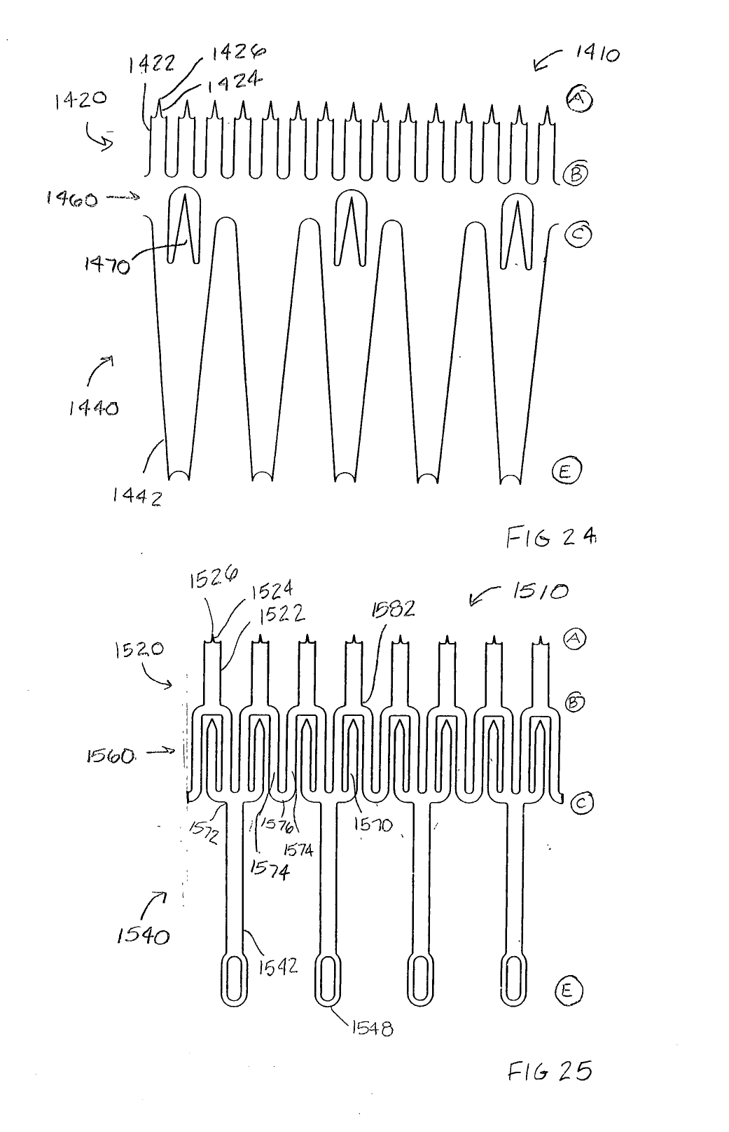

An illustrative embodiment of a connector 1410 in accordance with the invention is shown in FIG. 24. Connector 1410 may be described as including axially spaced first and second portions 1420 and 1440, respectively. First portion 1420 includes a plurality of annularly spaced members 1422. Members 1422 may be connected to members 1424 that have sharply pointed free end portions 1426 and that point away from second portion 1440. The ends of members 1422 that are closest to second portion 1440 are connected to medial portion 1460.

Second portion 1440 includes a plurality of annularly spaced members 1442. The ends of members 1442 that are closest to first portion 1420 are connected to medial portion 1460. Second portion 1440 includes a plurality of annularly spaced members 1470, which act as “stops” for back loading as described above in connection with FIG. 23. Members 1470 may point toward first portion 1420.

Connector 1410 has a cross-section that is similar to that shown in FIG. 13B because members 1424 point away from second portion 1440. Points A, B, C and E shown on connector 1410, in conjunction with the cross-sectional representation of a connector in FIG. 13B, demonstrate the approximate geometry that connector 1410 will assume after deployment.

An illustrative embodiment of a connector 1510 in accordance with the invention is shown in FIG. 25. Connector 1510 may be described as including axially spaced first and second portions 1520 and 1540, respectively. First portion 1520 includes a plurality of annularly spaced members 1522 that are connected to members 1524. Members 1524 may have sharply pointed free end portions 1526 and may point away from second portion 1540. The ends of members 1522 that are closest to second portion 1540 may be connected to medial portion 1560 at 1582.

Second portion 1540 includes a plurality of annularly spaced members 1542. The ends of members 1542 that are farthest from first portion 1520 may each have an attachment portion 1548. The ends of members 1542 that are closest to first portion 1520 may be connected to medial portion 1560 at 1572.

Medial portion 1560 may be annularly expandable. For example, medial portion 1560 may include a plurality of annularly spaced members 1574 that are connected to one another at 1576 and connected to first portion 1520 at 1582. Medial portion 1560 may also include members 1570, which act as “stops” for back loading as described above in connection with FIGS. 23-24. Members 1570 are connected to second portion 1540 at 1572.

Connector 1510 has a cross-section as shown in FIG. 13B because members 1524 point away from second portion 1540. Points A, B, C and E shown on connector 1510, in conjunction with the cross-sectional representation of a connector in FIG. 13B, demonstrate the approximate geometry that connector 1510 will assume after deployment.

An illustrative embodiment of a connector 1610 in accordance with the invention is shown in FIG. 26. Connector 1610 may be described as including axially spaced first and second portions 1620 and 1640, respectively. First portion 1620 includes a plurality of annularly spaced cells 1622. Cells 1622 may include annularly spaced members 1628 that are connected to one another at 1630 and 1632. First portion 1620 also includes a plurality of annularly spaced members 1624 that point toward second portion 1640 and that are connected to cells 1622 at 1630. Members 1624 may have sharply pointed free end portions 1626.

Second portion 1640 may include a plurality of annularly spaced members 1642. At the ends of members 1642 that are farthest from first portion 1620, each end may have an attachment portion 1648. The ends of members 1642 that are closest to first portion 1620 may be connected to medial portion 1660.

Medial portion 1660 may be annularly expandable. For example, members 1628 (a portion of which form cells 1622 of first portion 1620) may be connected to annularly adjacent members 1628 at medial portion 1660 by struts 1650. Thus, each individual cell 1622 may annularly enlarge (i.e., the spacing between each member 1628 that forms a cell 1622 may increase). Medial portion 1660 may also include members 1670, which act as “stops” for back loading as described above in connection with FIGS. 23-25.

Connector 1610 has a cross-section as shown in FIG. 13A because members 1624 point toward second portion 1640. Points A, B, C and E shown on connector 1610, in conjunction with the cross-sectional representation of a connector in FIG. 13A, show the approximate geometry that connector 1610 will assume after deployment. As shown, point A is located at free end portion 1626.

It will be understood that the foregoing is only illustrative of the principles of the invention, and that still other modifications can be made by those skilled in the art without departing from the scope and spirit of the invention. For example, the various materials and dimensions mentioned herein are only examples, and other materials and dimensions can be used, if desired.

The invention claimed is

1. A connector for use in making an anastomotic connection between a first aperture in a side wall of a graft conduit and a second aperture in a side wall of a body tissue conduit in a patient comprising a unitary structure disposed annularly about a longitudinal axis and having axially spaced first and second portions, the first portion having a plurality of annularly spaced first fingers that expand radially out to secure a perimeter of the first aperture to an exterior surface of the side wall of the body tissue conduit along a perimeter of the second aperture and having a plurality of annularly spaced members that have free ends configured to engage the side wall of the graft conduit, and the second portion having a plurality of annularly spaced second fingers that expand radially out to engage the side wall of the body tissue conduit.

2. The connector defined in claim 1 wherein the free ends of the annularly spaced members point away from the second portion.

3. The connector defined in claim 1 wherein the free ends of the annularly spaced members point toward the second portion.

4. The connector defined in claim 1 wherein the free ends of the annularly spaced members are sharply pointed.

5. The connector defined in claim 1 wherein a member of the plurality of annularly spaced members is connected to a first finger of the plurality of first fingers at the end of the first finger that is farthest from the second portion.

6. The connector defined in claim 1 wherein the structure has a medial portion between the axially spaced first and second portions.

7. The connector defined in claim 6 wherein at least one of the first portion, medial portion, and second portion is annularly enlargeable.

8. The connector defined in claim 7 wherein the annular enlargement of the at least one of the first portion, medial portion, and second portion is an elastic enlargement.

9. The connector defined in claim 6 wherein the medial portion has a fixed diameter.

10. The connector defined in claim 6 wherein the medial portion has a plurality of annularly spaced members that point toward the first portion to assist in positioning the connector with respect to the first aperture.

11. The connector defined in claim 1 wherein the second portion has a plurality of annularly spaced members that point toward the first portion to assist in positioning the connector with respect to the first aperture.

12. The connector defined in claim 1 wherein the radially outward expansion of the plurality of first fingers and the plurality of second fingers is an elastic expansion.

13. The connector defined in claim 1 wherein at least one of the plurality of second fingers has an attachment portion at the end of the at least one of the plurality of second fingers that is farthest from the first portion for attachment to a mold.

14. Apparatus for making an anastomotic connection between a first aperture in a side wall of a graft conduit and a second aperture in a side wall of a body tissue conduit in a patient comprising:
a tip structure having a substantially hemispherical distal end portion wherein the tip structure is configured for passage through the second aperture from outside the body tissue conduit;
a first tubular structure connected to the tip structure that extends proximally from the tip structure;
a substantially conical structure wherein the conical structure is disposed annularly around the first tubular structure;
a second tubular structure connected to the conical structure that extends proximally from the conical structure, and wherein the second tubular structure is disposed annularly around the first tubular structure; and
a third tubular structure wherein the third tubular structure is disposed annularly around the first and second tubular structures.

15. The apparatus defined in claim 14 wherein a diameter of the third tubular structure increases abruptly at a distal end portion of the third tubular structure.

16. The apparatus defined in claim 14 further comprising:
a hollow annular connector having a first portion and a second portion; and
wherein the tip structure is configured to shield at least a portion of the second portion of the connector.

17. The apparatus defined in claim 16 wherein the at least a portion of the second portion is constrained between the tip structure and the conical structure.

18. The apparatus defined in claim 14 further comprising:
a hollow annular connector having a first portion and a second portion; and
wherein the third tubular structure is configured to shield at least a portion of the first portion.

19. Apparatus for forming an aperture in a side wall of a graft conduit using a blade comprising:
a dilator structure having a substantially conical tip portion configured for passage through an open end of the graft conduit and a shaft portion extending proximally from the tip portion;
a tubular shaft structure extending from the proximal end of the dilator structure;
a tubular sheath disposed annularly around at least a portion of the dilator; and
a spring disposed annularly around the tubular shaft structure configured to control the movement of the dilator structure within the tubular sheath structure.

20. A method for making an anastomotic connection between a first aperture in a side wall of a graft conduit and a second aperture in a side wall of a body tissue conduit in a patient comprising:
introducing a hollow annular connector into the graft conduit so that at least a first axial portion of the connector is inside the graft conduit and a second axial portion of the connector extends from the graft conduit via the first aperture, and wherein at least a portion of the first axial portion is shielded during the introduction;
unshielding the at least a portion of the first axial portion;
approximating the first and second apertures so that the second axial portion of the connector extends from the first aperture into the second aperture, and wherein at least a portion of the second axial portion is shielded during the approximation; and
unshielding the at least a portion of the second axial portion so that the connector secures a perimeter of the first aperture to an exterior surface of the side wall of the body tissue conduit along a perimeter of the second aperture.

21. The method defined in claim 20 wherein the graft conduit has first and second segments that extend in respective opposite directions along the graft conduit from the first aperture, wherein body fluid will flow in the first segment after the connection has been made, wherein the second segment has a third aperture spaced from the first aperture, and wherein the introducing comprises:
inserting the connector into the second segment via the third aperture and passing the connector along the inside of the second segment to the first aperture.

22. The method defined in claim 21 further comprising closing the second segment after the connection has been made.

23. The method defined in claim 20 wherein the introducing comprises:
inserting the connector into the graft conduit via the first aperture.

24. The method defined in claim 20 wherein the first axial portion of the connector has a plurality of first fingers configured to engage the side wall of the graft conduit, and wherein the unshielding the at least a portion of the first portion comprises:
engaging the side wall of the graft conduit with the plurality of fingers.

25. The method defined in claim 20 wherein the first axial portion of the connector has a plurality of members having free ends configured to engage the side wall of the graft conduit, and wherein the unshielding the at least a portion of the first portion comprises:
engaging the side wall of the graft conduit with the free ends of the plurality of members.

26. The method defined in claim 20 wherein the second axial portion of the connector has a plurality of fingers configured to engage the side wall of the body tissue conduit, and wherein the unshielding the at least a portion of the second portion comprises:
engaging the side wall of the body tissue conduit with the plurality of fingers.
